(19) United States
(12) Patent Application Publication
(10) Pub. No.: US 2024/0044766 A1
(43) Pub. Date: Feb. 8, 2024

(54) NORMAL INCIDENT GUIDED-MODE-RESONANCE BIOSENSOR AND PROCALCITONIN DETECTION METHOD USING THE SAME

(71) Applicant: NATIONAL CHUNG CHENG UNIVERSITY, Chiayi County (TW)

(72) Inventors: GUO-EN CHANG, New Taipei City (TW); LAI-KWAN CHAU, Chiayi City (TW); YEN-SONG CHEN, Tainan City (TW); CHIA-JUI HSIEH, Chiayi City (TW)

(73) Assignee: NATIONAL CHUNG CHENG UNIVERSITY, Chiayi County (TW)

(21) Appl. No.: 18/355,418

(22) Filed: Jul. 20, 2023

(30) Foreign Application Priority Data
Aug. 2, 2022 (TW) 111129000

(51) Int. Cl. G01N 15/06; G01N 33/543

(52) U.S. Cl. CPC G01N 15/06; G01N 33/54346; G01N 2015/0693; G01N 2015/0065

(57) ABSTRACT

A normal incident guided-mode-resonance biosensor and procalcitonin detection method using the same are provided and include a light source, a first lens, a polarizer, a beam splitter, a ¼λ wave plate, a second lens, a detection unit, and a processing unit. The light source provides a light beam. The first lens converts the light beam into a parallel light. The polarizer filters and removes a transverse electric field mode light wave in the parallel light. The beam splitter selectively forms a transverse magnetic field mode light wave in the parallel light. The ¼λ wave plate rotates the transverse magnetic field mode light wave in the parallel light by 45°. The second lens focuses the transverse magnetic field mode light wave to the bio-sensing chip. The detection unit receives an emitted light of the bio-sensing chip and generates a sensing signal.

### CROSS-REFERENCE TO RELATED APPLICATION

This application claims priority from Taiwan Patent Application No. 111129000, filed on Aug. 2, 2022, in the Taiwan Intellectual Property Office, the content of which is hereby incorporated by reference in its entirety for all purposes.

### BACKGROUND OF THE INVENTION

### 1. Field of the Invention

The present disclosure relates to a technical field of optical biosensors, in particular to a normal incident guided-mode-resonance biosensor and a procalcitonin detection method using the same.

### 2. Description of the Related Art

Traditional medical tests have complex experimental steps that require a lot of time and reagents for inspectors. Under the circumstances of facing critical situations or waiting for a large number of samples to be tested, traditional medical tests are often insufficient to obtain test results. Therefore, the development of biochips has become one of the most important projects currently.

Sepsis is a disease in which the blood is infected with viruses, bacteria, and mold that cause fever, shock, and multiple organ failure, and severe sepsis has a mortality rate of up to 70%. Pathogenic bacteria and drug sensitivity require a period of blood incubation, so as to allow a certain number of pathogenic bacteria in the blood to be reached for testing. Due to the limitation of current medical tests in measuring samples, the treatment of sepsis is delayed, making the mortality rate remain high.

Accordingly, the inventor of the present disclosure has designed a biochip for rapid detection in an effort to tackle deficiencies in the prior art and further improve practical implementation in industries.

### SUMMARY OF THE INVENTION

In this view, to solve the aforementioned problems in the prior art, the present disclosure provides a normal incident guided-mode-resonance biosensor, including:
a light source, providing a light beam;
a first lens, disposed on an optical path of the light source to convert the light beam into a parallel light;
a polarizer, disposed relative to the first lens to filter and remove a transverse electric field mode light wave in the parallel light;
a beam splitter, disposed relative to the polarizer to selectively pass a transverse magnetic field mode light wave in the parallel light;
a ¼λ wave plate, disposed relative to the beam splitter to rotate the transverse magnetic field mode light wave in the parallel light by 45°;
a second lens, disposed relative to the ¼λ wave plate to focus the transverse magnetic field mode light wave into an incident light to a bio-sensing chip;
a detection unit, disposed relative to the beam splitter to receive an emitted light emitted from the bio-sensing chip and generate a sensing signal; and
a processing unit, electrically connected to the detection unit to receive and analyze the sensing signal;
wherein the emitted light is emitted into the detection unit through the second lens, the ¼λ wave plate, and the beam splitter, and the emitted light changes due to a change in a refractive index of a sample in the bio-sensing chip or an interaction with the sample.

Preferably, the bio-sensing chip includes:
a substrate;
a grating, disposed on the substrate, the grating diffracting the light beam for reflection to generate an emitted light; and
a waveguide layer, disposed on the grating, the waveguide layer adjusting a resonance wavelength after the light beam is incident.

Preferably, the bio-sensing chip further includes a plurality of metal nanoparticles fixed on one side of the grating, a detection antibody is modified on the plurality of metal nanoparticles, and the detection antibody has specificity to the target detection object, in order to bind the target detection object on the plurality of metal nanoparticles.

Preferably, a capture antibody is modified on the waveguide layer, and the capture antibody has specificity to the target detection object.

Preferably, the target detection object is procalcitonin.

Preferably, the bio-sensing chip further includes a runner, which is used to dispose the sample on the waveguide layer.

Preferably, a grating height of the bio-sensing chip is 40 to 70 nm, a thickness of the waveguide layer is 90 to 110 nm, and a resonance wavelength is 530 to 540 nm.

In addition, the present disclosure provides a method for detecting a target detection object, including steps as follows:
providing the normal incident guided-mode-resonance biosensor as mentioned above;
injecting a plurality of concentrations of the target detection object into the bio-sensing chip, and generating a standard sensing signal by the detection unit, which is sent to the processing unit;
calculating a calibration line through the standard sensing signal by the processing unit;
injecting a sample into the bio-sensing chip, and generating the sensing signal through the detection unit, which is sent to the processing unit; and
calculating a concentration of the target detection object through the sensing signal by the processing unit.

Preferably, the method for detecting the target detection object further include: mixing the sample with the plurality of metal nanoparticles modified with a detection antibody in order to bind the target detection object in the sample on the plurality of metal nanoparticles through the detection antibody.

Preferably, the processing unit calculates a concentration of the target detection object in the sample by Equation 1 as follows:

1−y=a+bx [Equation 1]

Wherein a is an intercept of the calibration line, b is a slope of the calibration line, x is the logarithmic (Log) value of the concentration of the target detection object in the sample, and y is the sensing signal.

Preferably, the target detection object is procalcitonin.

The efficacy of the present disclosure is that the normal incident guided-mode-resonance biosensor overcomes the overlapping problem of light sources and signals by using the principle of the polarization state of light, and excellent sensitivity and system stability may be obtained in conjunction with a bio-sensing chip having a grating period of 295 nm for the performance of an experiment on refractive index. The normal incident guided-mode-resonance biosensor may accurately detect subtle changes in refractive index, overcome the technical bottleneck of the traditional inspection method of limit detection concentration, and also quickly measure the concentration of the target detection object. This not only solves the technical problems of traditional inspection methods that require complicated procedures and a great deal of consumption in terms of time, manpower, and reagents, but also has the efficacy of being cost-effective, fast, and easy to operate. In the face of an emergency or a large number of samples waiting for testing, test results may be obtained in a short period of time.

The technical features of the present disclosure are to be illustrated in detail below with specific embodiments and accompanying drawings to make a person with ordinary skill in the art effortlessly understand the purposes, technical features, and advantages of the present disclosure.

### BRIEF DESCRIPTION OF THE DRAWINGS

The drawings required for the description of the embodiments of the present disclosure are to be briefly described below to illustrate more clearly the technical solutions of the embodiments of the present disclosure. It is obvious that the accompanying drawings described below are only some embodiments of the present disclosure. For a person with ordinary skill in the art, additional drawings can be obtained according to these drawings.

FIG. 1 is a schematic diagram of the normal incident guided-mode-resonance biosensor according to the present disclosure.

FIG. 2A is a reflectance spectrum of the structure of the bio-sensing chip with a period of 295 nm.

FIG. 2B is a linear fitting diagram of the resonance wavelength position and the refractive index.

FIG. 2C is a relational diagram of waveguide layer thickness, spectral sensitivity, and resonance wavelength position.

FIG. 2D is a comparative diagram of the energy distribution in the GMR structure with different waveguide thicknesses.

FIG. 3A is a relational diagram of grating height, sensitivity, and resonance wavelength position.

FIG. 3B is a relational diagram grating height, sensitivity, and evanescent wave energy in the GMR structure.

FIG. 4 is a relational diagram of incident light angle, sensitivity, and resonance wavelength position in the GMR structure.

FIG. 5 is a cross-sectional view of the substrate and grating of the bio-sensing chip according to the present disclosure.

FIG. 6 is a cross-sectional view of the bio-sensing chip in FIG. 2 after the formation of the waveguide layer.

FIG. 7 is a cross-sectional view of the bio-sensing chip in FIG. 3 after the formation of the runner.

FIG. 8 is a cross-sectional view of the bio-sensing chip in FIG. 3 after the connection with the catheter.

FIG. 9A is a schematic diagram of the completed assembly of the bio-sensing chip according to the present disclosure.

FIG. 9B is an SEM image of the bio-sensing chip according to the present disclosure.

FIG. 9C is an SEM image of gold nanoparticles modified on the waveguide layer.

FIG. 10 is a schematic diagram of the detection of the target detection object using metal nanoparticles of the bio-sensing chip according to the present disclosure.

FIG. 11A is the detection result of different concentrations of procalcitonin.

FIG. 11B is a calibration line for sample experiments performed three times on the purification of procalcitonin.

FIG. 12 is the real-time detection situation of the real sample of procalcitonin.

FIG. 13 is a flowchart of the method for detecting a target detection object by the normal incident guided-mode-resonance biosensor according to the present disclosure.

### DESCRIPTION OF THE PREFERRED EMBODIMENTS

The advantages, features, and technical methods of the present disclosure are to be explained in detail with reference to the exemplary embodiments and the figures for the purpose of being easier to be understood. Moreover, the present disclosure may be realized in different forms, and should not be construed as being limited to the embodiments set forth herein. Conversely, for a person with ordinary skill in the art, the embodiments provided shall make the present disclosure convey the scope more thoroughly, comprehensively, and completely. In addition, the present disclosure shall be defined only by the appended claims.

It should be noted that although the terms first, second, and the like may be used in the present disclosure to describe various elements, components, regions, sections, layers, and/or parts, these elements, components, regions, sections, layers and/or parts should not be limited by these terms. These terms are only used to distinguish one element, component, region, sections, layer, and/or part from another element, component, region, sections, layer, and/or part.

Unless otherwise defined, all terms (including technical and scientific terms) used in the present disclosure have the same meaning as those commonly understood by a person with ordinary skill in the art. It should be further understood that, unless explicitly defined herein, the terms such as those defined in commonly used dictionaries should be interpreted as having definitions consistent with their meaning in the context of the related art and the present disclosure, and should not be construed as idealized or overly formal.

### [Embodiment 1]: Framework of the Normal Incident Guided-Mode-Resonance Biosensor 1

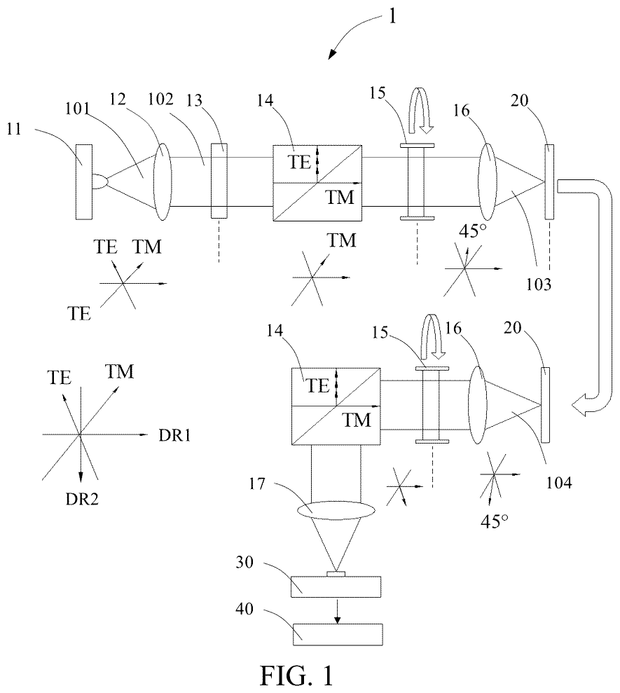

Please also refer to FIG. 1; FIG. 1 is a schematic diagram of the normal incident guided-mode-resonance biosensor 1 according to the present disclosure. The present disclosure provides a normal incident guided-mode-resonance biosensor 1, which includes: a light source, a first lens 12, a polarizer 13, a beam splitter 14, a ¼λ wave plate 15, and a second lens 16, sequentially disposed along an optical path of the light source (advancing direction of light) or in the first direction. That is, the first lens 12 is disposed on an optical path of the light source, the polarizer 13 is disposed relative to the first lens 12, the beam splitter 14 is disposed relative to the polarizer 13, the ¼λ wave plate 15 is disposed relative to the beam splitter 14, the second lens 16 is disposed relative to the ¼λ wave plate 15, in order to focus the transverse magnetic field mode light wave TM into an incident light 103 to a bio-sensing chip 20.

The light source may include, but are not limited to: LED lights.

To receive and analyze the emitted light 104 reflected from the bio-sensing chip 20, the normal incident guided-mode-resonance biosensor 1 further includes: a third lens 17, a detection unit 30, and a processing unit 40. The third lens 17 is disposed perpendicular to an optical path of the light source (advancing direction of light) or relative to the beam splitter 14 in a second direction, and the detection unit 30 is disposed relative to the third lens 17 to receive the emitted light 104 of the bio-sensing chip 20 and generate a sensing signal. The processing unit 40 is electrically connected to the detection unit 30 to receive and analyze the sensing signal. Wherein, the emitted light 104 is emitted into the detection unit 30 through the second lens 16, the ¼λ wave plate 15, and the beam splitter 14, and the emitted light 104 changes due to a change in a refractive index of a sample 220 in the bio-sensing chip 20 or an interaction with the sample 220.

Specifically, as shown in FIG. 1, a light beam 101 is emitted from the light source, the light is first converted into a parallel light 102 by the first lens 12, and then the transverse electric field mode light wave TE in the parallel light 102 is filtered by the polarizer 13, so that only the transverse magnetic field mode light wave TM enters and penetrates the beam splitter 14. The transverse magnetic field mode light wave TM is rotated 45° by the ¼λ wave plate 15 for the first time and then focused into an incident light 103 by the lens to enter the bio-sensing chip 20.

Next, the transverse magnetic field mode light wave TM is reflected back from the bio-sensing chip 20 to form the emitted light 104, and the emitted light 104 passes through the ¼λ wave plate 15 for the second time, so that the mode of the emitted light 104 is rotated by 45° again. At this moment, the emitted light 104 is rotated into a transverse electric field mode, enters the beam splitter 14, is reflected by 90°, and then is focused on the detection unit 30 through the third lens 17.

The normal incident guided-mode-resonance biosensor 1 of the present disclosure solves the problem of failing to receive the light signal by the traditional oblique incidence system after the normal incidence. Moreover, before the experiment, a spectrometer must be used to confirm the resonance wavelength position to make the resonance wavelength fall within the green light band to match the resonance peak of gold nanoparticles. Therefore, this step requires relevant knowledge, indicating that the operation is more difficult, so it is difficult for medical staff to operate independently. In addition, the normal incident guided-mode-resonance biosensor 1 of the present disclosure also solves the problem of the signal light source and original overlapping and filters out the background signal by a polarization splitter to enhance the system regularization sensitivity.

### [Experiment 2]: Optical Modeling of Bio-Sensing Chip 20

A simulation analysis is performed on the three parameters such as the grating 202 period, the waveguide layer 203 thickness, and the incident light 103 angle to find out the structure of the bio-sensing chip 20 that best fits the zero-degree angle reflective system. The grating 202 period is compared with two different periods, the waveguide layer 203 thickness is simulated from 80 nm to 130 nm, and the incident light 103 angle is an important parameter to control the resonance wavelength position to analyze an appropriate angle of the incident light 103 and its resonance wavelength.

Table 1 and Table 2 show the parameters of geometric modeling and material modeling:

TABLE 1

| Geometrical parameter | Numerical value |
|---|---|
| Grating 202 period (A) | 295 nm and 416 nm |
| Waveguide layer 203 depth (A) | 100 nm |
| Waveguide layer 203 thickness (t) | 80 to 130 nm |
| Incident light 103 angle (Q) | 0° and 14° |

TABLE 2

| | | Extinction |
|---|---|---|
| Material | Refractive index (n) | coefficient (k) |
| Target detection | 1.333 to 1.373 | 0 |
| object | | |
| TiO₂ | 4.74-9.85λ + 13.64λ + 6.44λ | 0 |
| COC substrate 201 | 1.68-0.58λ + 0.75λ + 0.34λ | 0 |
| AuNPs | −138.94 + 1060.41λ- | 34.97-184.54λ + |
| | 2925.21λ + 3499.27λ- | 329.39λ + 183.65λ |
| | 15040.99λ | |

### [Embodiment 3]: Optical Performance Analysis on the GMR Structure with the Grating 202 Period of 295 nm

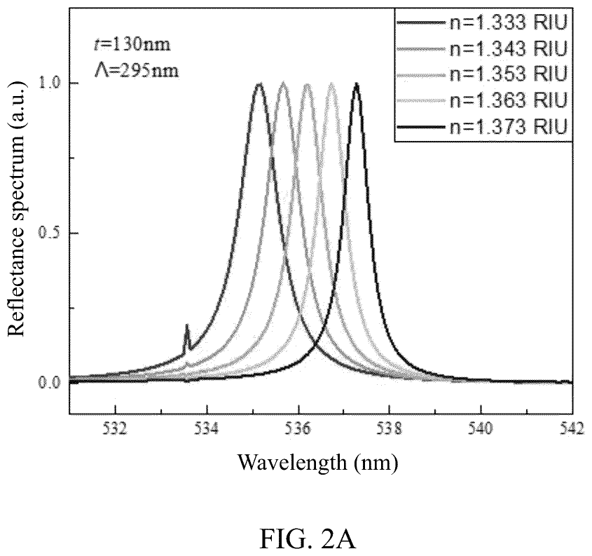

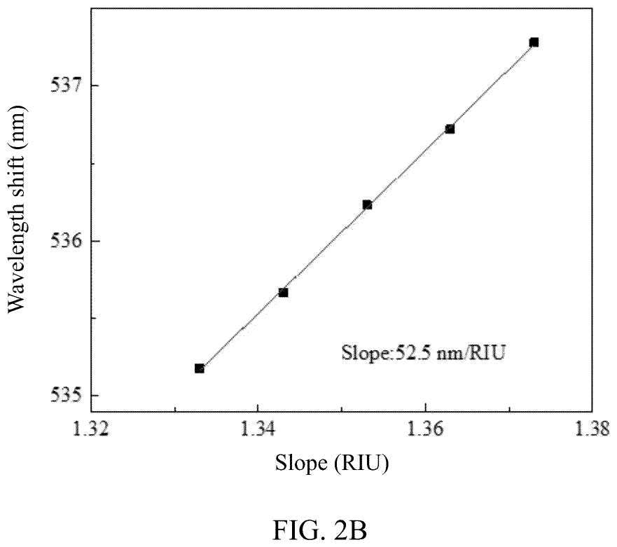

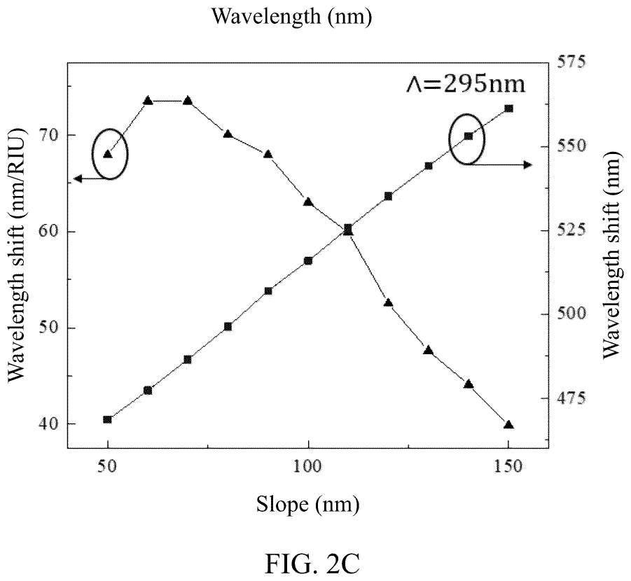

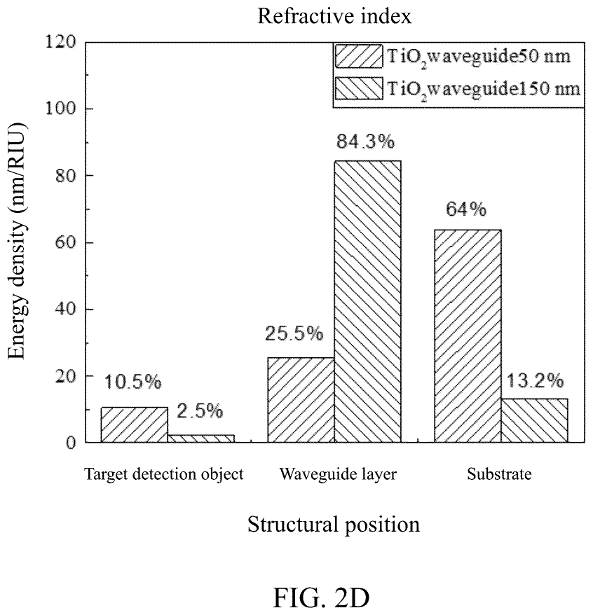

Please refer to FIG. 2A to FIG. 2D; FIG. 2A is a reflectance spectrum of the structure of the bio-sensing chip 20 with a period of 295 nm; FIG. 2B is a linear fitting diagram of the resonance wavelength position and the refractive index; FIG. 2C is a relational diagram of waveguide layer 203 thickness, spectral sensitivity, and resonance wavelength position; FIG. 2D is a comparative diagram of the energy distribution in the GMR structure with different waveguide thicknesses.

A waveguide mode resonance structure with the grating 202 period of 295 nm is produced by using nanoimprinting technology. The waveguide layer 203 thickness is an important parameter for the GMR biosensor, so the effects of different waveguide layer 203 thicknesses on the GMR biosensor with a period of 295 nm are to be discussed in this section. In the structure of the bio-sensing chip 20 with different waveguide layer 203 thicknesses, the refractive index of the test solution from 1.333 RIU to 1.373 RIU may be changed to obtain the reflectance spectrum shown in FIG. 2A. After the reflectance spectrum of five refractive indexes is obtained, linear fitting may be done with the refractive index and the resonance wavelength position. As shown in the linear fitting diagram of the reflection spectrum in FIG. 2B, the sensitivity of the GMR biosensor under the waveguide layer 203 thickness may be obtained with the equation.

As shown in FIG. 2C, with the waveguide layer 203 thickness increasing from 50 nm to 150 nm, the finest sensitivity is 73 nm/RIU at a thickness of 70 nm. Then, as the thickness increases, the sensitivity starts decreasing slowly when the waveguide layer 203 thickness is 150 nm, only 39.9 nm/RIU, which is 45.3% less than that when the waveguide layer thickness is 70 nm. The waveguide layer 203 thickness also affects the resonance wavelength position of the GMR structure. The research results show that the resonance wavelength position of the GMR structure would move to a long wavelength as the waveguide layer 203 thickness becomes thicker. When the waveguide layer 203 thickness increases from 50 nm to 150 nm, the resonance wavelength would move from 468 nm to 561 nm in a linear tendency. It may be concluded from the simulation result that the relationship between the waveguide layer 203 thickness, the sensitivity of the GMR structure, and the resonance wavelength position. In the future when designing the structure of the bio-sensing chip 20, this simulation result may be used to design the structure of the bio-sensing chip 20 that meets the requirements.

As shown in the energy distribution comparison diagram of different waveguide thicknesses in FIG. 2D, the result shows that when the waveguide layer 203 thickness is 150 nm and 50 nm, the evanescent wave energy in the structure respectively accounts for 2.5% and 10.5% of the total energy. This phenomenon explains why the sensitivity decreases when the waveguide layer 203 thickness is too thick. When the waveguide layer 203 is thicker, more energy will be coupled in the waveguide layer 203. In contrast, when the waveguide layer 203 is thinner, more energy will be coupled in the substrate 201. It may be known that the larger waveguide layer 203 thickness in the GMR structure does not necessarily mean better. Instead, adjustment is required according to different situations. For example, in this structure, the finest sensitivity may be obtained when the waveguide layer 203 thickness is 70 nm.

### [Example 4]: Grating 202 Height in the GMR Structure and Sensitivity Analysis

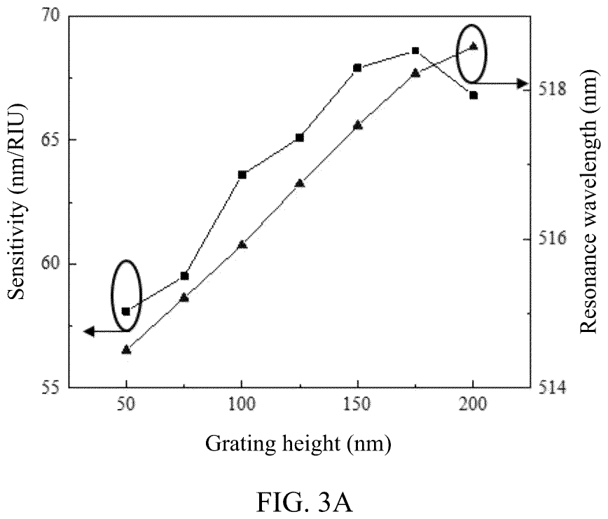

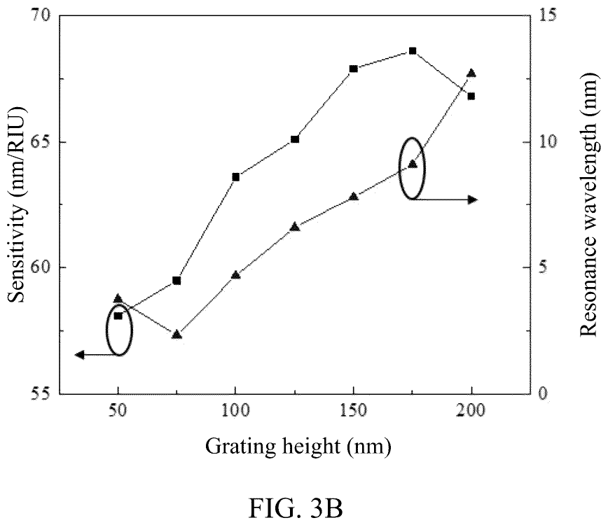

Please refer to FIG. 3A and FIG. 3B; FIG. 3A is a relational diagram of grating 202 height, sensitivity, and resonance wavelength position; FIG. 3B is a relational diagram of grating 202 height in the GMR structure, sensitivity, and evanescent wave energy. During the same period, the refractive index of the analyte is set to be 1.333 RIU to 1.373 RIU in the GMR structure with the grating 202 height ranging from 50 nm to 200 nm to analyze the relationship between the grating 202 height, the sensitivity, and the resonance wavelength position.

As shown in FIG. 3A, the square points and triangular points in the figure respectively represent the sensitivity and resonance wavelength position at each grating 202 height. It is found that the sensitivity of the GMR structure increases as the grating 202 height increases, and the finest sensitivity is found when the grating 202 height is 175 nm, which is 15.3% higher than that when the grating 202 height is 50 nm. However, the grating 202 height has little effect on the resonance wavelength position and only moves 4 nm when the height changes from 50 nm to 200 nm. Under the condition of the same period, as the grating 202 height increases, more evanescent waves would make contact with the test solution in the wave trough of the grating 202 structure, which accounts for the reason why the sensitivity increases as the grating 202 height increases.

As shown in FIG. 3B, in the relational diagram of sensitivity and evanescent wave energy, square points and triangular points respectively represent the sensitivity and evanescent wave energy of different grating 202 heights. When the grating 202 height increases from 50 nm to 200 nm, the sensitivity and evanescent wave energy will increase in a similar tendency. When the grating 202 height is 200 nm, the evanescent wave energy in the test solution is the largest, accounting for 12.96% of the total energy in the structure, which is 9% different from that when the grating 202 height is 50 nm. Accordingly, it may be further confirmed that when the grating 202 height changes, the evanescent wave energy in the test solution is one of the main reasons that affect the sensitivity of the GMR structure.

### [Embodiment 5]: Analysis of GMR of the Incident Light 103 Angle and the Grating 202 Period of 295 nm

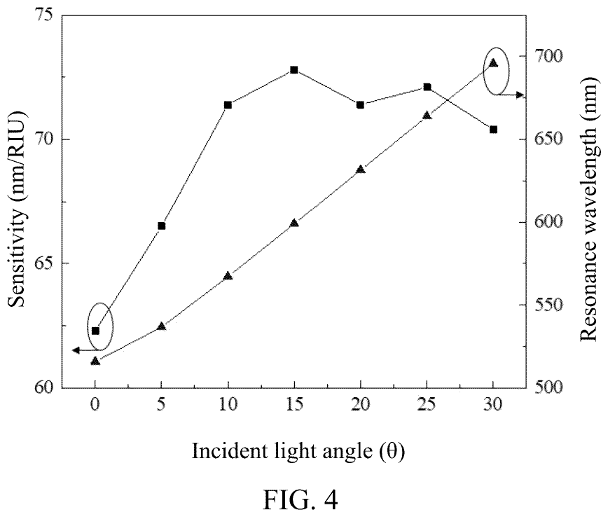

Please refer to FIG. 4; FIG. 4 is a relational diagram of the GMR structure of incident light 103 angle, sensitivity, and evanescent wave energy, and the square points and triangular points respectively represent the sensitivity and resonance wavelength position of the bio-sensing chip 20 when the incident light 103 is at an angle of 0° to 30°. It may be found from the simulation result that the sensitivity will increase as the incident light 103 angle becomes larger. When the incident light 103 angle is 15 degrees, the finest sensitivity of 72.8 nm/RIU may be obtained, which is 14.4% higher than that when the incident angle is 0 degree. When the incident angle exceeds 15 degrees, there is no significant increase in sensitivity. The resonance wavelength will increase with the incident light 103 angle, with a steady tendency to move toward the long wavelength. As the incident light 103 angle changes, the sensitivity and evanescent wave energy of the GMR structure will increase in a similar tendency and then start to decrease when the incident light angle is greater than 20 degrees. The intensity of the evanescent wave energy is why the sensitivity of the GMR structure increases as the incident light 103 angle increases. When the incident light 103 angle is rotated from 0° to 30°, the resonance wavelength will move from 515 nm to 695 nm. Since 695 nm is close to the edge of the visible light band, the analysis is not continued for larger incident light 103 angles.

As can be seen, through Embodiments 2 to 4, the following results are obtained using the finite element analysis software:
1. The waveguide layer 203 thickness affects the contact area between the evanescent wave and the test solution, which in turn affects the sensitivity of the guided-mode-resonance biosensor.
2. The waveguide layer 203 thickness affects the resonance wavelength position in the GMR structure, and the thicker the waveguide layer 203 is, the more the resonance wavelength will be moved to a long wavelength.
3. For the normal incident guided-mode-resonance biosensor, since the working wavelength ranges from 540 nm to 560 nm, the acceptable error angle is 4°.
4. The incident light 103 angle affects the sensitivity of the guided-mode-resonance biosensor, and the sensitivity of light intensity is better at normal incidence than at oblique incidence, and the spectral sensitivity is better at oblique incidence than at normal incidence.
5. The grating 202 height has a slight effect on the sensitivity of the GMR structure with a period of 295 nm but has almost no effect on the resonance wavelength position.
6. The incident light 103 angle has a certain effect on the sensitivity and resonance wavelength of the GMR structure with a period of 295 nm, and the finest spectral sensitivity is obtained at an incident light 103 angle of 20 degrees.
7. The grating 202 period has a great effect on the sensitivity and quality factor of the GMR structure and the finest spectral sensitivity may be obtained in the visible band when the grating 202 period is 466 nm.
8. It is found through the simulation software that the main reason for the sensitivity change when changing the structural parameters of the GMR is the contact area between the evanescent wave and the test solution.
9. The normal incident system may not only reduce the difficulty of system operation but also increase the light intensity of the guided-mode-resonance biosensor.

This present disclosure reduces the difficulty of system operation by improving the grating 202 period of the bio-sensing chip 20 and developing a normal incident system. Since the grating 202 period used in the past is 416 nm, the incident light 103 angle must be at 14° of resonance wavelength to be located at 540 nm, in order to be in coordination with gold nanoparticles for experiments. Therefore, the present disclosure uses COMSOL finite element analysis software to redesign the grating 202 period and the waveguide layer 203 thickness to allow the resonance wavelength to fall at 540 nm after the normal incidence of the light source. After simulation, it is confirmed that the resonance wavelength of the structure of the bio-sensing chip 20 with the waveguide layer 203 thickness at 130 nm at a period of 295 nm falls at about 537 nm when the light source is incident in the normal direction.

### [Embodiment 6]: Preparation of Bio-Sensing Chip 20

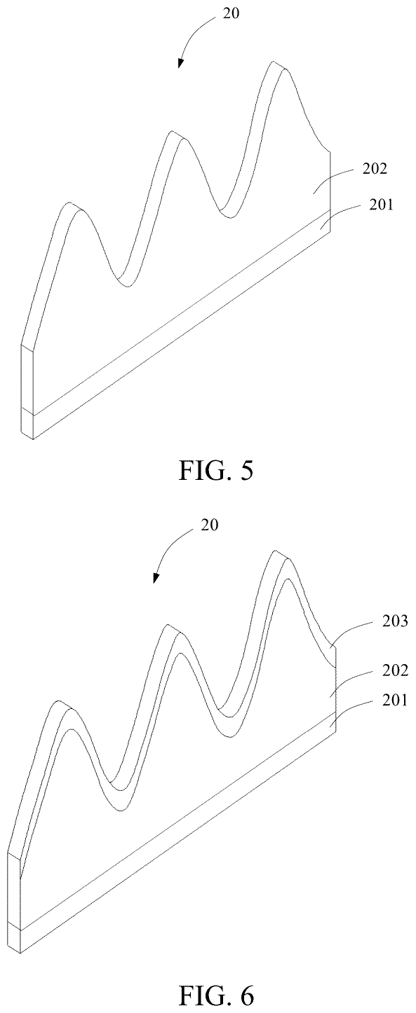

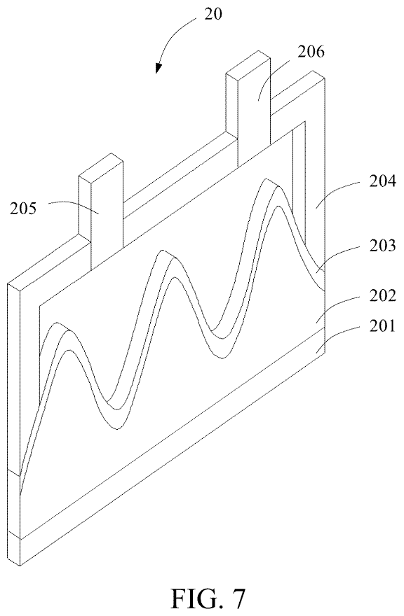

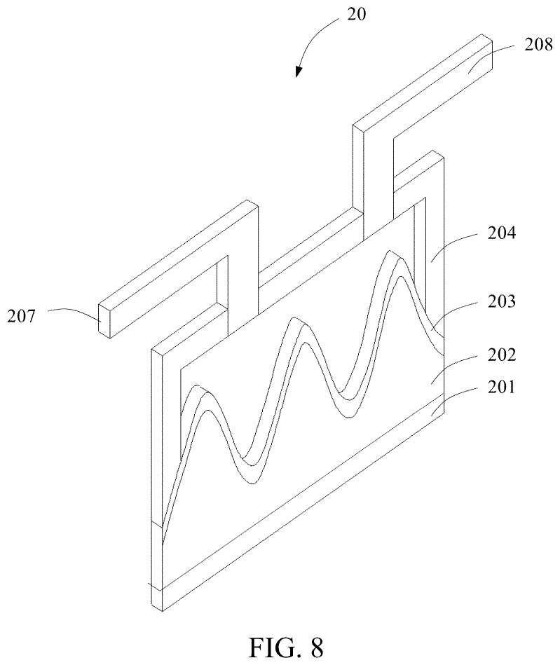

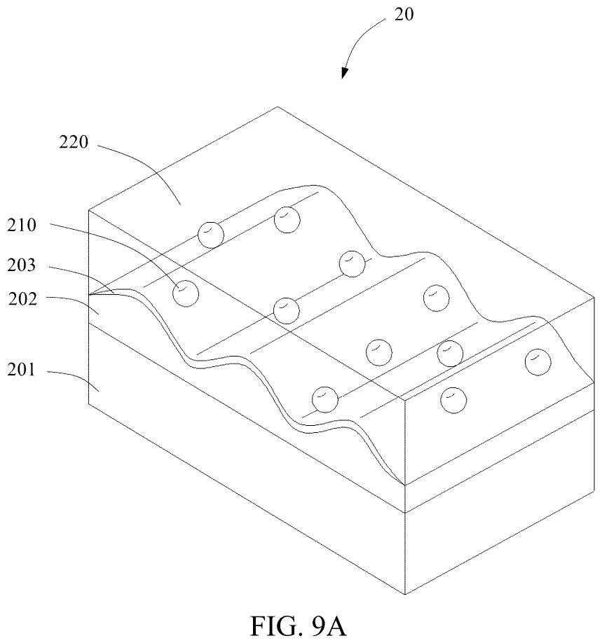

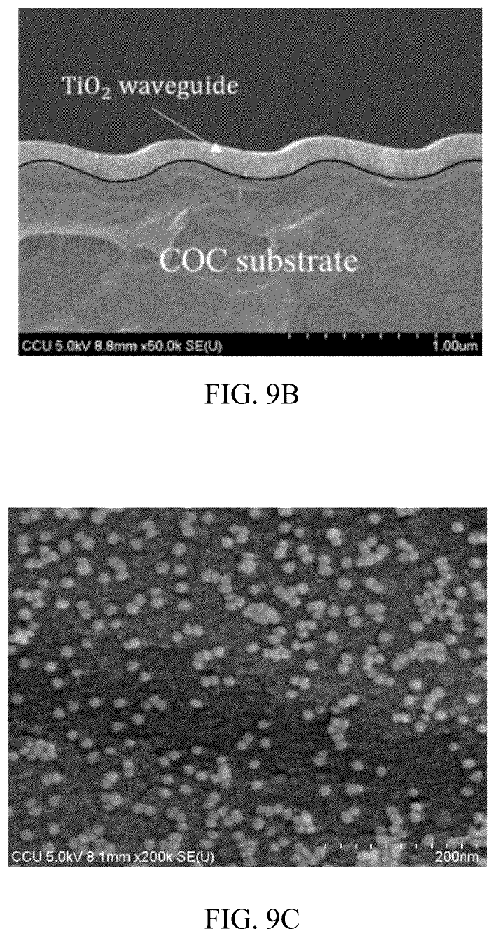

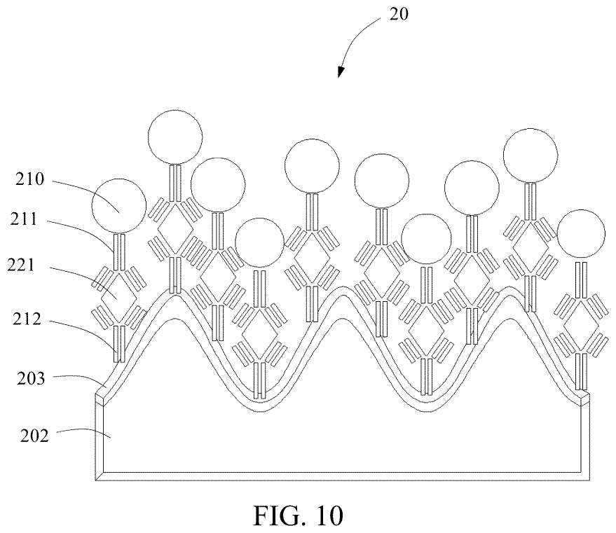

Please refer to FIG. 5 to FIG. 9B; FIG. 5 is a cross-sectional view of the substrate 201 and grating 202 of the bio-sensing chip 20 according to the present disclosure; FIG. 6 is a cross-sectional view of the bio-sensing chip 20 in FIG. 2 after the formation of the waveguide layer 203; FIG. 7 is a cross-sectional view of the bio-sensing chip 20 in FIG. 3 after the formation of the runner; FIG. 8 is a cross-sectional view of the bio-sensing chip 20 in FIG. 3 after the connection with the catheter; FIG. 9A is a schematic diagram of the completed assembly of the bio-sensing chip 20 according to the present disclosure; FIG. 9B is an SEM image of the bio-sensing chip 20 according to the present disclosure.

As shown in FIG. 9A, the bio-sensing chip 20 of the present disclosure is a GMR biochip, which includes: a substrate 201, a waveguide layer 203, a grating 202, a runner, and a plurality of metal nanoparticles 210. The grating 202, the runner, and the substrate 201 are made of cyclic olefin copolymers (COC) and are made by an injection molding machine. The grating 202 diffracts the light beam 101 to reflect and generate an emitted light 104, the waveguide layer 203 adjusts the resonance wavelength of the incident light beam 101, and the runner disposes the sample 220 on the waveguide layer 203.

As shown in FIG. 5 to FIG. 9B, the manufacturing process of the bio-sensing chip 20 is presented as follows: A grating 202 is formed on the substrate 201, and a layer of TiO₂ waveguide layer 203 is plated on the grating 202 by using an evaporation machine. The waveguide layer 203 thickness may be adjusted by the feedback signal from the quartz oscillator inside the machine through the rate at which TiO₂ is plated on the substrate, and the membrane thickness is initially measured using the frequency change of the quartz oscillator (Crystal). When the membrane thickness reaches the set thickness parameter, the plating process will be stopped to achieve time control, and each batch of plated grating 202 pieces will be measured by a rough surface plating instrument to confirm the plating thickness. Then, double-sided tape is used to glue the runner cover 204 and the grating 202. Lastly, the charge-in pipe 207 and the charge-out pipe 208 for injecting the test solution are glued to the runner inlet 205 and runner outlet 206 of the runner cover 204 respectively with AB glue to complete the manufacturing process of the bio-sensing chip 20.

In the present embodiment, the grating 202 height is 40 to 70 nm, the waveguide layer 203 thickness is 90 to 110 nm, and the resonance wavelength is 530 to 540 nm.

### [Embodiment 7]: Preparation of Metal Nanomaterials

Since the molecular weight of the target detection object is very small, e.g., the molecular weight of procalcitonin is about 12.7 kD, and the limit of detection (LOD) of the guided-mode-resonance biosensor is about 7×10⁻⁷ g/ml, it is not easy to detect the biological response of procalcitonin. Therefore, in the present study, metal nanoparticles 210 are used together with Sandwich ELISA and two antibodies to specifically identify the antigen 221 of the target detection object. The biological response and sensing signal generated when the procalcitonin (PCT) is bound is enhanced to make the intensity of the emitted light 104 directly proportional to the concentration of the target detection object.

In the present embodiment, three different solutions will be used. The first solution is the phosphate-buffered saline (PBS) solution, which is made by mixing a specific proportion of Na₂HPO₄, NaCl, KCl, and KH₂PO₄ with deionized water (DI Water) as a solvent. Because the pH value of PBS solution is similar to that of human blood, many research teams use this solution as a buffer solution. In the present embodiment, the solution is also used to wash away the gold nanoparticles not bonded to the bio-sensing chip 20 and detection antibody 211 to ensure experimental accuracy.

The second solution is metal nanoparticles 210, which is the gold nanoparticle solution in the present embodiment. The antibody modified on the gold nanoparticles is mixed with the test solution and injected into the bio-sensing chip 20 to amplify the signal variation when the antigen and antibody are bound. Both the shape and size of gold nanoparticles affect their physical properties. Please refer to FIG. 9C; FIG. 9C is an SEM image of gold nanoparticles modified on the waveguide layer 203. The darker part in the SEM image is the wave trough of the grating 202 structure, the lighter part is the peak, and the evenly distributed circular particles are spherical gold nanoparticles with a diameter of 13 nm. The gold nanoparticles used in the present embodiment are spherical gold nanoparticles with a diameter of 13 nm, which are synthesized by adding sodium citrate to tetrachloroaurate solution.

The third solution is the antigen of procalcitonin (PCT), which is used as the target detection object in the present embodiment, mixed with the gold nanoparticle solution modified with procalcitonin antibodies, and injected into the bio-sensing chip 20 of Embodiment 6 for detection.

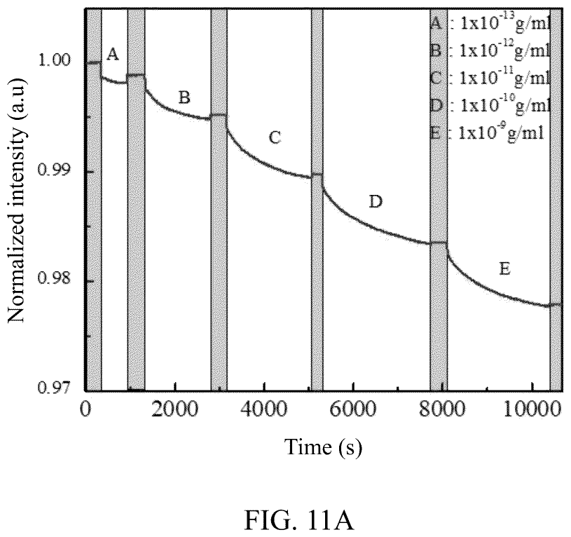

Please refer to FIG. 10; FIG. 10 is a schematic diagram of the detection of the target detection object using metal nanoparticles 210 of the bio-sensing chip 20 according to the present disclosure. In this modification method, the target detection object is procalcitonin antigen (PCT), the procalcitonin antibody is used as the detection antibody 211 and the capture antibody 212 respectively, and the metal nanoparticles 210 are gold nanoparticles. Firstly, the capture antibody 212 is modified on the bio-sensing chip 20, and the detection antibody 211 is modified on the gold nanoparticles and then mixed with the target detection object, in order to allow the procalcitonin antigen and the detection antibody on the gold nanoparticles 211 to form a bond, which is finally injected into the bio-sensing chip 20 for the observation of the biological signal generated by the binding of the analyte and the capture antibody 212 as the sensing signal. After the capture antibody 212 on the waveguide layer 203 captures the target detection object bound to the plurality of metal nanoparticles 210, the intensity of the emitted light 104 from the bio-sensing chip 20 is changed, thus passing through the detection unit 30 to generate a sensing signal.

### [Embodiment 8]: Analysis of the Establishment of Calibration Line and Limit of Detection of Procalcitonin

The experimental steps of procalcitonin (PCT) purification sample 220 are as follows:
1. Modify the procalcitonin antibody (Capture Antibody) on the bio-sensing chip 20.
2. Confirm whether or not the gold nanoparticle solution (herein referred to as solution A) modified with procalcitonin antibody (Detection antibody) has the phenomenon of aggregation.
3. Adjust five different concentrations of procalcitonin antigen (herein referred to as solution B).
4. Mix solution A and solution B, letting them stand for 15 minutes, and waiting for the antibody and antigen to complete bonding.
5. Inject the buffer solution into the bio-sensing chip 20 and wait for the system to stabilize.
6. Inject the test solution mixed in step 4 into the bio-sensing chip 20 and wait for the bonding reaction to complete (RSD less than 7×10⁻⁵ indicates that the signal is stable).
7. Inject buffer solution to wash away unbonded gold nanoparticles.
8. Inject the test solution of the next concentration.
9. Repeat steps 6, 7, and 8 until five different test concentrations are completed.
10. Record the experimental data and calculate the limit detection concentration.

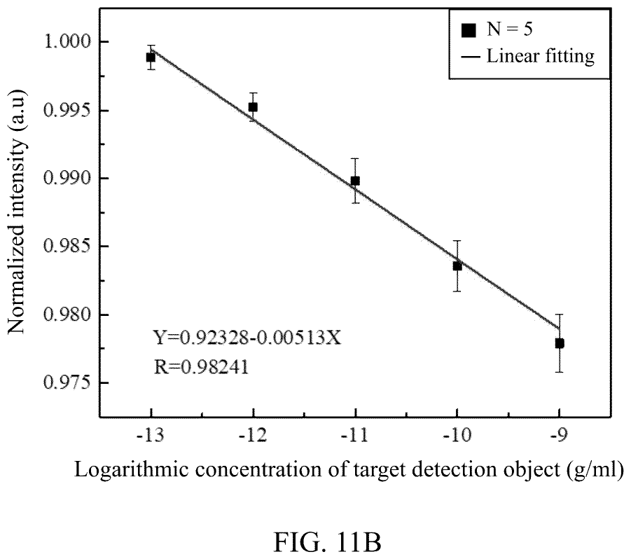

According to the above experimental steps, a procalcitonin (PCT) real-time detection diagram is obtained. Please refer to FIG. 11A; FIG. 11A is the detection result of different concentrations of procalcitonin. A to E are the detection results of five standards with different concentrations. PBS solution is injected between the two concentrations to wash away the gold nanoparticles that are not bonded to the bio-sensing chip 20. The standard sensing signal is generated by the detection unit 30, and after the signal is stable, the next concentration is injected for detection. As can be seen from FIG. 11A, when five standards with different concentrations were tested, the system is able to detect significant biological curves, and as the detection standard concentration increases, the biological response tendency also increases.

In the present embodiment, multiple concentration experiments are used to establish a calibration line. It can be found in FIG. 11A that when the test solution is injected, the light intensity will slowly decrease and finally level off. The calibration line is drawn by taking the light intensity of PBS for 100 seconds on average after each detection concentration. Please refer to FIG. 11B; FIG. 11B is a calibration line for sample experiments performed three times on the purification of procalcitonin. After linear fitting by the processing unit 40, the intercept and slope of the calibration line may be obtained, which may be introduced into the limit of detection (LOD) of Equation 2 to calculate the limit of detection (LOD) of the present experiment.

1−3σᵣ=a+bx [Equation 2]

where a is an intercept of the calibration line, b is a slope of the calibration line, x is a logarithm of the limit of detection, and σᵣ is the relative standard deviation.

After calculation, it may be obtained that the limit detection concentration of the bio-sensing chip 20 in the present study is 4.2×10⁻¹⁴ g/ml. In addition, the concentration of sample A 220 in FIG. 5 to FIG. 6 is 1×10⁻¹³ g/ml, which is very close to the calculated LOD, and an obvious biological response curve may be observed. This phenomenon may prove that the limit detection concentration calculated by Equation 2 is accurate, and since the concentration of procalcitonin PCT in normal human blood ranges from 0.02 ng/ml to 100 ng/ml, the limit detection concentration of 2.06×10⁻¹⁴ g/ml is quite sufficient for the detection of sepsis. The present embodiment confirms that the guided-mode-resonance biosensor, together with the sandwich method, may indeed be used to detect sepsis.

### [Embodiment 8]: Measurement and Analysis of Procalcitonin

A real blood plasma sample 220 containing procalcitonin (PCT) is tested on an angle-adjustable system, proving that the guided-mode-resonance biosensor is able to detect real sample 220, and the experimental steps are as follows:
1. Perform a multi-concentration experiment of purification sample 220 to establish a calibration line.
2. Determine the dilution multiple of the blood plasma sample 220 according to the detection range of the calibration line.
3. Mix the diluted blood plasma sample 220 with the nanogold solution modified with the procalcitonin antibody as the test solution.
4. Inject the buffer solution into the chip, wait for the system to stabilize, and then inject the test solution.
5. Wait for the biological response curve to stabilize (representing the end of the biological response).
6. Record the data, which is then introduced into the calibration line to calculate the original concentration.

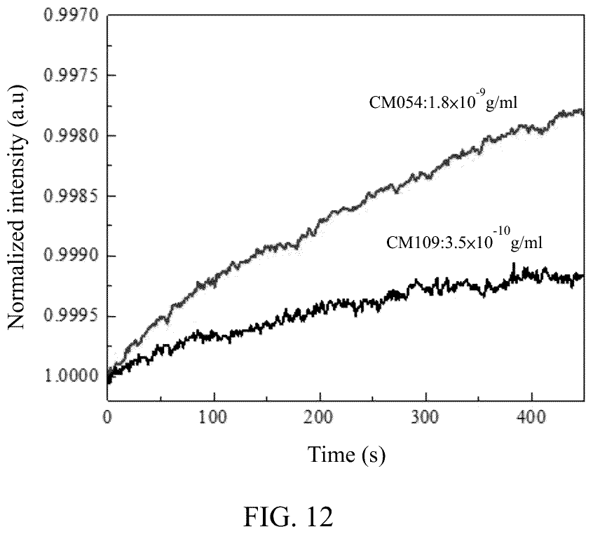

Please refer to FIG. 12; FIG. 12 is the real-time detection situation of the real sample of procalcitonin (The sample 220 concentration is 3.5×10⁻¹⁰ g/ml and 1.8×10⁻⁹ g/ml). These two concentrations respectively represent the concentrations of procalcitonin in the blood during severe infection and mild sepsis. From the figure, obvious biological response signals may be seen when different concentrations of the real sample 220 are injected. Through careful observation, it can be found that the biological response curve generated when injecting the real sample 220 with a higher concentration is also more significant. When 300 seconds are reached, the signal of the blood plasma sample 220 for mild sepsis infection subsided, while the signal of the plasma sample 220 for severe sepsis infection continues to rise. This phenomenon confirms that the guided-mode-resonance biosensor may indeed distinguish different concentrations of procalcitonin (PCT) in the blood plasma sample 220, and sepsis infection may be detected within five minutes. Next, the average intensities of the two buffer solutions before and after the test solution will be subtracted as the signal variation generated by the test solution, which is then introduced into the calibration line to calculate the original concentration.

After the signal variation generated by the real sample 220 is obtained from the data, it may be introduced into Equation 1 to calculate the original concentration of the detection sample 220. Since the concentration of the sample 220 will be diluted within the range of the calibration line before the real sample 220 is tested, the concentration of the sample 220 calculated by the equation needs to be multiplied by the dilution multiple. Table 3 shows the detection result of the real sample 220. After the comparison of the data in the table, it is found that with the different original concentrations of the sample 220, the detection concentration detected by the system also has the same tendency. However, the differential multiple ratios between the detected concentration and the original concentration are presumed to be that the real sample 220 has been stored for too long, which is caused by a decrease in the concentration of procalcitonin protein in sample 220.

In the equation of the calibration line, y is the signal variation of the real sample 220, a and b respectively are the intercept and slope of the calibration line, and x is the Log value of 220 concentration of the real sample after dilution.

1−y=a+bx [Equation 1]

Where a is an intercept of the calibration line, b is a slope of the calibration line, x is the logarithm (Log) value of the concentration of the target detection object in the sample 220, and y is the sensing signal.

TABLE 3

| Sample | Original | Detection | Dilution | Differential |
|---|---|---|---|---|
| code | concentration | concentration | multiple | multiple ratios |
| CM054 | 1.8 × 10⁻⁹ g/ml | 1.01 × 10⁻¹⁰ g/ml | 200 | 17 |
| CM109 | 3.5 × 10⁻¹⁰ g/ml | 1.68 × 10⁻¹¹ g/ml | 200 | 20 |
| CM141 | 2.53 × 10⁻¹⁰ g/ml | 9.46 × 10⁻¹² g/ml | 2 × 107 | 26.74 |
| INF001 | 2.97 × 10⁻⁹ g/ml | 4.6 × 10⁻¹¹ g/ml | 2 × 107 | 64. |
| CM135 | 1.22 × 10⁻⁹ g/ml | 1.3 × 10⁻¹¹ g/ml | 2 × 107 | 93.84 |

In the present embodiment, the experiment of the multiple procalcitonin (PCT) blood plasma samples is carried out on the guided-mode-resonance biosensor, and the original concentration of the sample is calculated by using the calibration line. Since the concentration of the blood plasma sample to be tested must be within the range of the calibration line, the blood plasma sample will be diluted before the detection is performed. From Table (5-1), it is found that the concentration difference calculated by the more diluted blood plasma sample will be larger, while the calculated concentration of the sample diluted by fewer times is closer to the original concentration. The reason why there is a difference between the detection concentration and the original concentration is due to excessive storage time for drugs and errors in diluting the drugs.

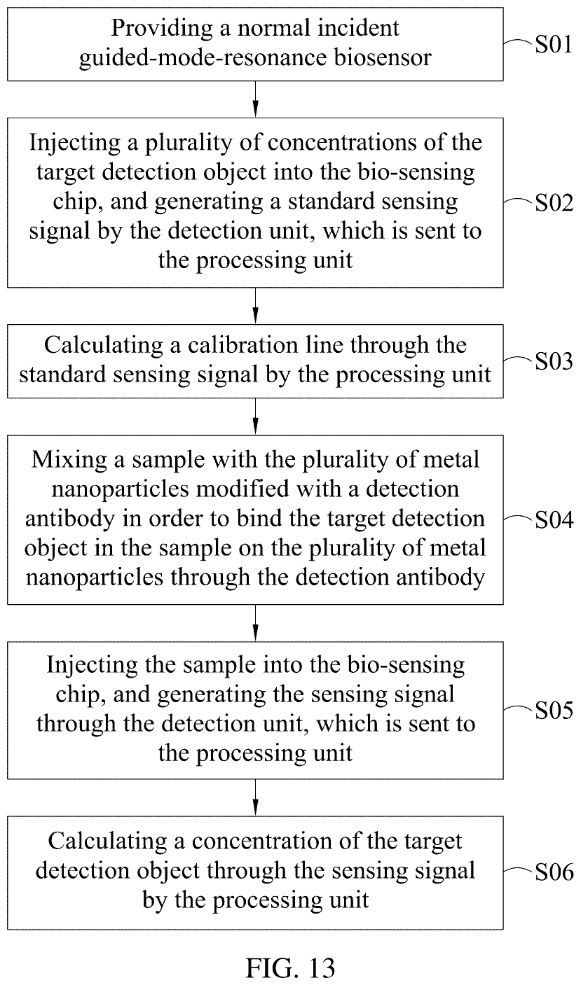

Please refer to FIG. 13; FIG. 13 is a flowchart of the method for detecting a target detection object by the normal incident guided-mode-resonance biosensor 1 according to the present disclosure. Based on the aforementioned embodiments, the present disclosure further provides a method for detecting a target detection object, including the steps as follows:
Step S01: Providing a normal incident guided-mode-resonance biosensor 1;
Step S02: Injecting a plurality of concentrations of the target detection object into the bio-sensing chip 20, and generating a standard sensing signal by the detection unit 30, which is sent to the processing unit 40;
Step S03: Calculating a calibration line through the standard sensing signal by the processing unit 40;
Step S04: Mixing a sample 220 with the plurality of metal nanoparticles 210 modified with a detection antibody 211 in order to bind the target detection object in the sample 220 on the plurality of metal nanoparticles 210 through the detection antibody 211;
Step S05: Injecting the sample 220 into the bio-sensing chip 20, and generating the sensing signal through the detection unit 30, which is sent to the processing unit 40; and
Step S06: Calculating a concentration of the target detection object through the sensing signal by the processing unit 40.

The details of the aforementioned steps are as described in the above multiple embodiments and shall not be repeated herein.

### [Embodiment 9]: Table of Procalcitonin (PCT) Biosensor Performance Comparison

Table 4 is a table of performance comparison between the normal incident guided-mode-resonance biosensor 1 of the present disclosure and other systems. Various biosensors for the detection of procalcitonin are shown in the table, including electrochemical immunoassay (ECIA), fiber optic nanogold-linked immunosorbent assay (FONLISA), and the guided-mode-resonance biosensor used in the present disclosure. The limit detection concentration can reach very low, but the waveguide mode biosensor used in the present disclosure may detect sepsis infection in only 5 minutes, which is a great advantage for detecting sepsis, a disease with high urgency.

TABLE 4

| | Limit of | | | |
|---|---|---|---|---|
| | detection | | | |
| | (LOD) | Time | | |
| Method | pg/ml | (min) | | Reference |
| PETIA | 53 | 10 | | AM Dupuy (2020) |
| ECIA | 0.03 | >70 | min | Vashist, S. K et al.(2016) |
| SPR | 2500 | 10 | min | F. Battagliab et al.(2021) |
| FONLISA | 0.095 | 15 | min | CY Chiang et al.(2020) |
| ICA | 3 | 15 | min | Taranova, N. A. et al. |
| | | | | (2017) |
| CLIA | 3.9 | <30 | min | G. Wang (2020) |
| Normal | 0.042 | <5 | min | The present disclosure |
| incident | | | | |
| guided-mode- | | | | |
| resonance | | | | |
| biosensor | | | | |

The detection unit of the present disclosure may be any device, optical sensor, or photoelectric sensor capable of detecting light intensity, for example, Si Detector of Thorlabs, model: DET36A2, to convert a light signal into an electric signal, which is sent to a processing unit.

The processing unit of the present disclosure may be any device or application with computing capabilities and may be built into any detection unit or into a separate computer, and the processing unit of the present disclosure may also be built into a computer as an application program.

The efficacy of the present disclosure is that the normal incident guided-mode-resonance biosensor overcomes the overlapping problem of light sources and signals by using the principle of the polarization state of light, and excellent sensitivity and system stability may be obtained in conjunction with a bio-sensing chip having a grating period of 295 nm for the performance of an experiment on refractive index. The normal incident guided-mode-resonance biosensor may accurately detect subtle changes in refractive index, overcome the technical bottleneck of the traditional inspection method of limit detection concentration, and also quickly measure the concentration of the target detection object. This not only solves the technical problems of traditional inspection methods that require complicated procedures and a great deal of consumption in terms of time, manpower, and reagents, but also has the efficacy of being cost-effective, fast, and easy to operate. In the face of an emergency or a large number of samples waiting for testing, test results may be obtained in a short period of time.

The above description is merely illustrative rather than restrictive. Any equivalent modifications or alterations without departing from the spirit and scope of the present disclosure are intended to be included in the following claims.

What is claimed is:

1. A normal incident guided-mode-resonance biosensor, comprising:
a light source, providing a light beam;
a first lens, disposed on an optical path of the light source to convert the light beam into a parallel light;
a polarizer, disposed relative to the first lens to filter and remove a transverse electric field mode light wave in the parallel light;
a beam splitter, disposed relative to the polarizer to selectively pass a transverse magnetic field mode light wave in the parallel light;
a ¼λ wave plate, disposed relative to the beam splitter to rotate the transverse magnetic field mode light wave in the parallel light by 45°;
a second lens, disposed relative to the ¼λ wave plate to focus the transverse magnetic field mode light wave into an incident light to a bio-sensing chip;
a detection unit, disposed relative to the beam splitter to receive an emitted light emitted from the bio-sensing chip and generate a sensing signal; and
a processing unit, electrically connected to the detection unit to receive and analyze the sensing signal;
wherein the emitted light is emitted into the detection unit through the second lens, the ¼λ wave plate, and the beam splitter, and the emitted light changes due to a change in a refractive index of a sample in the bio-sensing chip or an interaction with the sample.

2. The normal incident guided-mode-resonance biosensor according to claim 1, wherein the bio-sensing chip comprises:
a substrate
a grating, disposed on the substrate, the grating diffracting the light beam for reflection to generate an emitted light; and
a waveguide layer, disposed on the grating, the waveguide layer adjusting a resonance wavelength after the light beam is incident.

3. The normal incident guided-mode-resonance biosensor according to claim 2, wherein the bio-sensing chip further comprises a plurality of metal nanoparticles fixed on one side of the grating, a detection antibody is modified on the plurality of metal nanoparticles, and the detection antibody has specificity to the target detection object, in order to bind the target detection object on the plurality of metal nanoparticles.

4. The normal incident guided-mode-resonance biosensor according to claim 3, wherein a capture antibody is modified on the waveguide layer, and the capture antibody has specificity to the target detection object.

5. The normal incident guided-mode-resonance biosensor according to claim 2, wherein the target detection object is procalcitonin.

6. The normal incident guided-mode-resonance biosensor according to claim 2, wherein the bio-sensing chip further comprises a runner, which is used to dispose the sample on the waveguide layer.

7. The normal incident guided-mode-resonance biosensor according to claim 2, wherein a grating height of the bio-sensing chip is 40 to 70 nm, a thickness of the waveguide layer is 90 to 110 nm, and a resonance wavelength is 530 to 540 nm.

8. A method for detecting a target detection object, comprising steps as follows:
providing the normal incident guided-mode-resonance biosensor according to claim 1;
injecting a plurality of concentrations of the target detection object into the bio-sensing chip, and generating a standard sensing signal by the detection unit, which is sent to the processing unit;
calculating a calibration line through the standard sensing signal by the processing unit;
injecting a sample into the bio-sensing chip, and generating the sensing signal through the detection unit, which is sent to the processing unit; and
calculating a concentration of the target detection object through the sensing signal by the processing unit.

9. The method according to claim 8, further comprising: mixing the sample with the plurality of metal nanoparticles modified with a detection antibody in order to bind the target detection object in the sample on the plurality of metal nanoparticles through the detection antibody.

10. The method according to claim 8, wherein the processing unit calculates a concentration of the target detection object in the sample by Equation 1 as follows:
1−y=a+bx [Equation 1]
Wherein a is an intercept of the calibration line, b is a slope of the calibration line, x is the logarithmic (Log) value of the concentration of the target detection object in the sample, and y is the sensing signal.

11. The method according to claim 8, wherein the target detection object is procalcitonin.
